(19) United States
(12) Patent Application Publication
(10) Pub. No.: US 2024/0180821 A1
(43) Pub. Date: Jun. 6, 2024

(54) ORALLY DISINTEGRATING FILM COMPOSITION OF KETOROLAC AND A METHOD OF PREPARATION THEREOF

(71) Applicant: LN LABORATORIES PVT LTD, Hyderabad (IN)

(72) Inventors: Gunneswara Subrahmanya Vara Prasad RANGABHATLA, Hyderabad (IN); Venkatanarayana KURELLA, Hyderabad (IN)

(21) Appl. No.: 18/283,807

(22) PCT Filed: Mar. 24, 2022
(86) PCT No.: PCT/IB2022/052710

§ 371 Date: Sep. 24, 2023

(30) Foreign Application Priority Data
Mar. 24, 2021 (IN) 202141012896

(51) Int. Cl. A61K 9/00; A61K 31/407; A61K 47/02; A61K 47/10; A61K 47/12; A61K 47/26; A61K 47/32; A61K 47/38; A61K 47/40

(52) U.S. Cl. CPC A61K 9/006; A61K 31/407; A61K 47/02; A61K 47/10; A61K 47/12; A61K 47/26; A61K 47/32; A61K 47/38; A61K 47/40

(57) ABSTRACT

An orally disintegrating film composition of ketorolac is disclosed. The composition comprises of therapeutically effective concentration of ketorolac of about 10%-20% by total weight of the composition and one or more pharmaceutically acceptable excipients that include solubilizers, polymer, plasticizer, disintegrating agent, sweetening agent, cooling agent, flavouring agent, pH modulating agents, colouring agent and purified water.

### FIELD OF INVENTION

The present invention relates to oral disintegrating film and the process of manufacturing the same. It relates more specifically to oral disintegrating film of Ketorolac and its salts

### BACKGROUND OF THE INVENTION

Ketorolac tromethamine is a member of the pyrrolo-pyrrole group of nonsteroidal anti-inflammatory drugs (NSAIDs). The chemical name for ketorolac tromethamine is (±)-5-Benzoyl-2,3-dihydro-1H-pyrrolizine-1-carboxylic acid, compound with 2-amino-2-(hydroxymethyl)-1,3-propanediol, and the structural formula is:

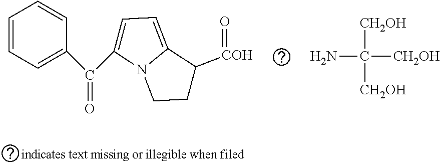

C₁₅H₁₃NO₃·C₄H₁₁NO₃

Ketorolac tromethamine is a racemic mixture of [−]S and [+]R ketorolac tromethamine. Ketorolac tromethamine may exist in three crystal forms. All forms are equally soluble in water. Ketorolac tromethamine has a pKa of 3.5 and an n-octanol/water partition coefficient of 0.26. The molecular weight of ketorolac tromethamine is 376.41.

Tablets are most preferred oral dosage forms because of its convenience in terms of self-administration and accurate dosing. However, the problem of this dosage form is poor patient compliance particularly by the geriatric and pediatric patients who experience difficulty in swallowing. Rapidly disintegrating dosage form, particularly one that disintegrates and dissolves/disperses in saliva and can be administered without need of water, is convenient and appreciated. Ketorolac tromethamine is susceptible to significant degradation in aqueous compositions. The drug is known to undergo chemical degradation via oxidation, hydroxylation and other unknown routes. The oxidative degradation product reported is 1-keto analogue, and the hydroxylation degradation product reported is 1-hydroxy analogue. Aqueous and ethanolic solutions of ketorolac or salts thereof are susceptible to degradation when subjected to long term storage under room temperature (PCT application no's. WO 2009/087658 and WO 20121127497). Apart from this, ketorolac tromethamine is bitter in taste, and adequate degree of palatability is needed especially for a formulation meant for administration through oral cavity.

Orally disintegrating Films (ODF) or sublingual technologies have drawn attention in the industry as a promising approach to deliver drugs for a wide range of the patient population. ODF can be prepared by various processes. This technique employs super disintegrants like croscarmellose sodium, directly compressible diluents, sweeteners and flavoring agents. The other important factors to be considered in the preparation of an ODF include effective taste-masking and to provide acceptable taste upon oral disintegration.

The stability of the ODF dosage forms can be challenging depending upon drugs and excipients used in the formulations. It is hard to achieve stable ODFs, as most of the ingredients used in preparing ODFs dissolve in with minimum quantity of water. Therefore, a careful identification and selection of excipients is critical for the development and long-term stability of the tablets.

Conventional oral ketorolac tablets which are available in the market are not suitable for acute inflammatory conditions where quick onset of action is required. Particularly due to its use as an analgesic, a quick onset of action of ketorolac assumes a significant importance. Multiple attempts were made to provide quick onset of action of ketorolac by formulating it in different possible forms. However, the stability and taste-masking of these formulations appear to be challenging factors, as ketorolac is susceptible to oxidative and hydrolytic degradation, and is bitter in taste leading to poor compliance. When in contact with mouth or throat mucosa, an unpleasant taste is felt. There is a need to mask/inhibit the bitter taste of ketorolac given in disintegrated form during oral use such as syrup, effervescent tablet, oral disintegrating tablet. Even today, there is patient's complaining about bitter taste left in their mouth or throat after administering ketorolac effervescent tablets.

Oral dissolving films are gaining interest as an alternative to fast dissolving tablets. The films are designed to dissolve upon contact with a wet surface, such as the tongue, within a few seconds, meaning the consumer can take the product without need for additional liquid. This convenience provides both a marketing advantage and increased patient compliance. As the drug is directly absorbed into systemic circulation, degradation in gastrointestinal tract and first pass effect can be avoided. These points make this formulation most popular and acceptable among pediatric and geriatric patients and patients with fear of choking. Over-the-counter films for pain management and motion sickness are commercialized in the US markets.

The oral route is one of the most preferred routes of drug administration as it is more convenient, cost effective, and ease of administration lead to high level of patient compliance. The oral route is problematic because of the swallowing difficulty for pediatric and geriatric patients who are afraid of choking. Patient convenience and compliance-oriented research has resulted in bringing out safer and newer drug delivery systems. Recently, fast dissolving drug delivery systems have started gaining popularity and acceptance as one such example with increased consumer choice, for the reason of rapid disintegration or dissolution, self-administration even without water or chewing as to overcome swallowing difficulties associated with tablets and capsules for pediatric and geriatric patients. The surface of buccal cavity comprises stratified squamous epithelium which is essentially separated from the underlying tissue of lamina propria and submucosa by an undulating basement membrane. It is interesting to note that the permeability of buccal mucosa is approximately 4-4,000 times greater than that of the skin, but less than that of the intestine.

An ideal fast dissolving delivery system should have the following properties: High stability, transportability, ease of handling and administration, no special packaging material or processing requirements, no water necessary for application, and a pleasant taste. Therefore, they are very suitable for pediatric and geriatric patients; bedridden patients; or patients suffering from dysphagia, parkinson's disease, Mucositis, or vomiting. Formulation of fast dissolving buccal film involves material such as strip-forming polymers, plasticizers, active pharmaceutical ingredients, sweetening agents, saliva stimulating agent, flavoring agents, coloring agents, stabilizing and thickening agents, permeation enhancers, and super disintegrants.

### PRIOR ART

U.S. Pat. No. 8,216,610 discloses the swallow formulation comprising paracetamol which facilitates the rapid delivery of paracetamol into the circulatory system following oral administration.

Patent No: EP3781145A1 discloses a fast-dissolving film composition containing paracetamol whose bitter taste is masked by using a mixture of sodium carbonate, citric acid, and magnesium alumino metasilicate as masking agents.

WO2016151461A1 discloses the preparation of sublingual films comprising Ketorolac (2-10 mg) with pharmaceutically acceptable excipients with a diffusion flux of 10-110 μg/cm²/min through biological mucosal membranes equivalent to human oral mucosa.

WO2004087111 discloses the preparation of rapidly disintegrating formulation for oral taste masking compositions where coating is insoluble or non-swellable in the pH range that exists in the mouth thereby disintegrating in the stomach. Patent No. EP0421 825 discloses the rally administrable solution comprising an analgesic as active ingredient and a solvent therefore which solvent contains polyethylene glycol.

PCT application NO. WO2016024928 discloses the taste masked oral pharmaceutical formulations where active ingredient is paracetamol and metallic salts are used.

Patent application CN1679525 discloses the acetaminophen orally disintegrating tablet.

Rutesh H. Dave et al, (Development and evaluation of high loading oral dissolving film of aspirin and acetaminophen) discloses the development and evaluation of physicochemical properties of acetaminophen and aspirin containing oral disintegrating strips with high loading dose.

Oral disintegrating film as disclosed in above prior art does not provide the desirable film properties. The present invention aims to provide oral disintegrating film and process for manufacturing the same. It relates more specifically to oral disintegrating film containing ketorolac.

### BRIEF SUMMARY OF THE INVENTION

In one object, the present invention provides herein, oral disintegrating film composition consisting essentially of a therapeutically effective amount of ketorolac and an excipient that facilitates oral administration, and methods of use thereof for antipyretic for children.

In another object, the present invention provides oral disintegrating film composition comprising ketorolac and their respective salts, solubilizers, polymer, plasticizer, disintegrating agent, sweetening agent, flavouring agent, pH modulating agents, colouring agent and purified water.

### DETAILED DESCRIPTION OF THE INVENTION

The present invention provides herein, compositions consisting essentially of a therapeutically effective amount of ketorolac and pharmaceutically acceptable excipients that facilitates oral administration and process for manufacturing the same.

In a preferred embodiment, the pharmaceutical composition of the invention is oral disintegrating film.

In the most preferred embodiment, the pharmaceutical oral disintegrating film composition comprising ketorolac and pharmaceutically acceptable excipients.

ketorolac is preferably used in a pharmaceutical oral disintegrating film composition of about 10% to about 20% based on the total weight of the composition. Most preferred concentration of ketorolac in the composition is about 10% to about 20% by the total weight of the composition.

In another embodiment, the pharmaceutical oral disintegrating film composition comprising ketorolac, solubilizers, polymer, plasticizer, disintegrating agent, sweetening agent, flavouring agent, pH modulating agents, colouring agent and purified water.

In yet another embodiment, the ketorolac pharmaceutical oral disintegrating film of the present invention is characterized in that the raw material components of the ketorolac oral disintegrating film include by total weight of composition contains ketorolac 10%-20% and solubilizers 0.1%-10%, polymer 10%-25%, plasticizer 0.1%-10%, disintegrating agent 1%-20%, sweetening agent 0.33%-4%, flavouring agent 2.5%-10%, pH modulating agents 0.05%-5%, colouring agent 0.001%-0.1% and purified water preferably used in the pharmaceutical oral disintegrating composition of sufficient quantity.

Examples of the solubilizer is selected from the group consisting of benzyl alcohol, hydroxypropyl betacyclodextrin, macrogol 15 hydroxystearate, polyoxyethylene alkyl ethers, polyoxyethylene castor oil derivatives (kolliphor RH 40), povidone, sulfobutylether betacyclodextrin, tricaprylin, triolein, polyoxyethylene sorbitan fatty acid esters, polyethylene-propylene glycol copolymers and any combinations thereof. Preferably, the solubilizer is selected from hydroxypropyl betacyclodextrin. Solubilizer preferably used in the pharmaceutical oral disintegrating film composition of about 0.1% to about 10% based on the total weight of the composition. Most preferably, solubilizer is used in the composition of about 1% to about 5% based on total weight of the composition.

Examples of the polymer is selected from the group consisting of hydroxyl propyl methyl cellulose, ethyl cellulose, hydroxypropyl cellulose, hydroxyl ethyl cellulose, polyvinyl alcohol, cellulose acetate phthalate, hydroxyl propyl methyl cellulose phthalate or any other cellulose derivative, polysaccharide derivatives and any combinations thereof. Preferably, the polymer selected is hydroxyl propyl methyl cellulose. Polymer preferably used in the pharmaceutical oral disintegrating film composition of about 10% to about 25% based on the total weight of the composition. Most preferably, polymer is used in the composition of about 12% to about 20% based on total weight of the composition.

Examples of the plasticizer is selected from the group consisting of polyethylene glycol 400, polyethylene glycol 4000, polyethylene glycol 6000, polyethylene glycol 8000, diethyl phthalate, propylene glycol, castor oil, triacetin, triethyl citrate, tributyl citrate, acetyl tributyl citrate, acetyl triethyl citrate, trioctyl citrate, acetyl trioctyl citrate, trihexyl citrate, dibutyl sebacate, polyvinyl pyrrolidone K-30, glycerol, sorbitol and any combinations thereof. Preferably, the plasticizer is selected from glycerol. Plasticizer preferably used in the pharmaceutical oral disintegrating film composition of about 0.1% to about 10% based on the total weight of the composition. Most preferably, plasticizer is used in the composition of about 1% to about 6% based on total weight of the composition.

Examples of the disintegrating agent is selected from the group consisting of crospovidone, microcrystalline cellulose, crosscarmellose sodium, Polacrilin potassium, sodium starch glycolate, pregelatinzed starch, carboxymethyl cellulose, silicified microcrystalline cellulose and modified corn starch and any combinations thereof. Preferably, the disintegrating agent is selected silicified microcrystalline cellulose. Disintegrating agent preferably used in the pharmaceutical oral disintegrating film composition of about 1% to about 20% based on the total weight of the composition. Most preferably, disintegrating agent is used in the composition of about 5% to about 15% based on total weight of the composition.

Examples of the sweeting agent is selected from the group consisting of artificial sweeteners like aspartame, sugars derivatives, sucralose, dextrose, fructose, isomalt, lactilol, maltitol, ammonium glycyrrhizinate, maltose, mannitol, sorbitol, Aspartame, sodium saccharine, Acesulfame Potassium, Stevioside, starch hydrolysate, polydextrose and xylitol and any combinations thereof. Preferably, the sweetening agents are selected from sucralose, xylitol and ammonium glycyrrhizinate. Sweetening agents preferably used in the pharmaceutical oral disintegrating film composition of about 0.33 to about 4% based on the total weight of the composition. Most preferably, sweetening agent is used in the composition of about 0.3% to about 4% based on total weight of the composition.

Examples of the flavour agents are selected from the group consisting of peppermint flavour, cooling flavour (menthol), flavour oils, flavouring aromatic oil, peppermint oil, clove oil, bay oil, anise oil, eucalyptus oil, thyme oil, oil of bitter almonds. Flavouring agents include, vanilla, chocolate flavour, citrus oils, fruit essences, and any combinations thereof. Preferably, the flavour agents are selected from menthol and chocolate flavour. Flavour agents preferably used in the pharmaceutical oral disintegrating film composition of about 2.5% to about 10% based on the total weight of the composition. Most preferably, flavouring agents are used in the composition of about 3% to about 7% based on total weight of the composition.

Examples of the pH modulating agents is selected from the group consisting of sodium carbonate, ammonium carbonate, sodium bicarbonate, potassium bicarbonate, calcium carbonate, magnesium carbonate, disodium glycine carbonate, sodium glycine carbonate, lysine carbonate, arginine carbonate, citric acid, tartaric acid, malic acid, fumaric acid, metatartaric acid, adipic acid, sodium acid citrate, ascorbic acid and amino acids such as aspartic acid, glutamic acid, glycine, leucine, tyrosine and tryptophan and any combinations thereof. Preferably, the pH modulating agents are selected from sodium carbonate and citric acid. pH modulating agents preferably used in the pharmaceutical oral disintegrating film composition of about 0.05% to about 5% based on the total weight of the composition. Most preferably, pH modulating agent is used in the composition of about 0.5% to about 5% based on total weight of the composition.

Examples of the colouring agent is selected from the group consisting of sunset yellow, amaranth, red iron oxide, natural juice concentrates, pigments and opacifying agents such as titanium oxide, silicon dioxide and zinc oxide, solid choco color and any combinations thereof. Preferably, the coloring agent is selected from solid choco color. Colouring agent preferably used in the pharmaceutical oral disintegrating film composition of about 0.001% to about 0.1% based on the total weight of the composition. Most preferably, colouring agent is used in the composition of about 0.01% to about 0.08% based on total weight of the composition.

The following example is provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Example 1

Oral disintegrating film with following solvent system composition

| S. No. | Ingredients | w/w % |
|---|---|---|
| 1 | Ketorolac | 10%-20% |
| 2 | Sucralose | 0.33%-4% |
| 3 | Silicified microcrystalline cellulose | 1%-20% |
| 4 | Hydroxypropyl Beta-cyclodextrin | 0.1%-10% |
| 5 | Ammonium Glycyrrhizinate | 0.33%-4% |
| 6 | Xylitol | 0.33%-4% |
| 7 | Hydroxyl Propyl Methyl Cellulose | 10%-25% |
| 8 | Glycerol | 0.1%-10% |
| 9 | Flavor | 2.5%-10% |
| 10 | Cooling Agent Flavor | 2.5%-10% |
| 11 | Sodium Carbonate | 0.05%-5% |
| 12 | Citric Acid | 0.05%-5% |
| 13 | Color | 0.001%-0.1% |
| 14 | Purified Water | Q.S to 100% |

| | |
|---|---|
| Total strip weight | 100.00% |

### Example-2

| S. No. | Ingredients | % w/w |
|---|---|---|
| 1 | Ketorolac Tromethamine | 11.11-22.22 |
| 2 | Sucralose | 3.34-6.67 |
| 3 | Maltodextrin | 5.44-10.89 |
| 4 | Pregelatinized hydroxypropyl pea starch | 3.34-6.67 |
| 5 | Hydroxypropyl Methylcellulose | 17.78-35.56 |
| 6 | Glycerol | 6.67-13.33 |
| 7 | Tartrazine | 0.11-0.22 |
| 8 | Peppermint flavour | 2.22-4.44 |
| 9 | Purified Water* | q.s. |
| | Total | 100.00% |

*water get evaporated during the coating prosses and traces of water content will remain product.

### Manufacturing Procedure

1. Weighed quantity of purified water was taken in a ss container and kept under stirring at 350 RPM.
2. Weighed quantity of ketorolac tromethamine was added to above step-1 under stirring at 350 RPM for 5 min.
3. Weighed quantity of sucralose was added to above step-2 under stirring at 350 RPM for 3 min.
4. Weighed quantity of maltodextrin was added to above step-3 under stirring at 350 RPM for 3 min.
5. Weighed quantity of Pregelatinized hydroxypropyl pea starch was added to above step-4 under stirring at 350 RPM for 3 min.
6. Weighed quantity of 11% HPMC solution was added to the above step-5 under stirring at 500 rpm for 10 mins.
7. Weighed quantity of Tartrazine was added to above step-6 under stirring at 500 RPM for 3 min.
8. Weighed quantity of glycerol was added to above step-7 under stirring at 500 RPM for 2 min.
9. Weighed quantity of peppermint Flavour was added to above step-8 under stirring at 500 RPM for 3 min.
10. Finally, above step-9 Wet slurry was stirred at 500 RPM for 20 min to get uniform dispersion.
11. Above step wet slurry after completion of mixing subjected to vacuum degassing for 15 mins to obtain bubble free slurry.
12. The wet slurry was Casted at process parameters of Dr. Knife thickness of 400 to 500 microns; Temperature 90±3° C.; In-direct drying for 15 min.
13. The dried casted film was Slitted into 25 mm×40 mm for 20 mg dose.
14. The dried casted film was slitted into 25 mm×20 mm for 10 mg dose.

The films were evaluated for disintegration time, folding endurance, Assay Dissolution, and impurities. The films were subjected to stability study under 40° C.±2° C. and 75%±5% RH condition for one month and evaluated above test parameters and results are tabulated.

### Results:

| | | | Results | |
|---|---|---|---|---|
| | | | | After |
| S. No. | Test Parameters | Limits | Initial | 1 month |
| 1 | Disintegrating time | NMT 60 sec | 18 sec | 20 sec |
| 2 | Folding Endurance | NLT 10 | 46 | 40 |
| 3 | Assay | 90-110% | 102.42 | 101.86 |
| 4 | Dissolution | NLT 75(Q)% | 98.68 | 98.22 |
| | Impurities: | | | |
| 5 | Impurity A | NMT 0.5% | 0.14 | 0.16 |
| | Impurity B | NMT 0.5% | ND | ND |
| | Impurity C | NMT 0.8% | 0.02 | 0.08 |
| | Impurity D | NMT 0.5% | ND | ND |
| S. No. | Ingredients | | | % w/w |
| 1 | Ketorolac tromethamine | | | 8.62-17.24 |
| 2 | Beta cyclodextrin | | | 8.62-17.24 |
| 3 | Sucralose | | | 2.58-5.17 |
| 4 | Maltodextrin | | | 4.22-8.45 |
| 5 | Pregelatinized hydroxypropyl pea starch | | | 2.58-5.17 |
| 6 | Hydroxypropyl Methylcellulose | | | 13.79-27.59 |
| 7 | Glycerol | | | 5.17-10.34 |
| 8 | Tartrazine colour | | | 0.08-0.17 |
| 9 | Peppermint Flavour | | | 1.72-3.45 |
| 10 | Orange Flavour | | | 2.59-5.18 |
| 11 | Purified Water | | | q.s. |
| Total | | | | 100.00% |
| | Unknown max | NMT 0.5% | 0.07 | 0.13 |
| | Impurity | | | |
| | Total Impurities | NMT 1.0% | 0.23 | 0.37 |

ND: Not detected

### Example-3

* water get evaporated during the coating prosses and traces of water content will remain product.

### Manufacturing Procedure

1. Weighed quantity of purified water was taken in a ss container and kept under stirring at 350 RPM.
2. Weighed quantity of ketorolac tromethamine was added to above step-1 under stirring at 350 RPM for 5 min.
3. Weighed quantity of Beta-cyclodextrin was added to above step-2 under stirring at 350 RPM for 5 min.
4. Weighed quantity of sucralose was added to above step-3 under stirring at 350 RPM for 3 min.
5. Weighed quantity of maltodextrin was added to above step-4 under stirring at 350 RPM for 3 min.
6. Weighed quantity of Pregelatinized hydroxypropyl pea starch was added to above step-5 under stirring at 350 RPM for 3 min.
7. Weighed quantity of 11% HPMC solution was added to the above step-6 under stirring at 500 rpm for 10 mins.
8. Weighed quantity of Tartrazine was added to above step-7 under stirring at 500 RPM for 3 min.
9. Weighed quantity of glycerol was added to above step-8 under stirring at 500 RPM for 2 min.
10. Weighed quantity of peppermint Flavour was added to above step-9 under stirring at 500 RPM for 3 min.
11. Weighed quantity of Orange Flavour was added to above step-10 under stirring at 500 RPM for 3 min.
12. Finally, above step-11 Wet slurry was stirred at 500 RPM for 20 min to get uniform dispersion.
13. Above step wet slurry after completion of mixing subjected to vacuum degassing for 15 mins to obtain bubble free slurry.
14. Wet slurry was casted at process parameters of Dr. Knife thickness of 500 to 600 microns; Temperature 90±3° C.; In-direct drying for 15 min.
15. The dried casted film was Slitted into 25 mm×40 mm for 20 mg dose.
16. The dried casted film was slitted into 25 mm×20 mm for 10 mg dose.

The films were evaluated for disintegration time, folding endurance, Assay Dissolution, and impurities. The films were subjected to stability study under 40° C.±2° C. and 75%±5% RH condition for one month and evaluated above test parameters and results are tabulated.

### Results:

| | | | Results | |
|---|---|---|---|---|
| S. | | | | After |
| No. | Test Parameters | Limits | Initial | 1 month |
| 1 | Disintegrating time | NMT 60 sec | 15 sec | 18 sec |
| 2 | Folding Endurance | NLT 10 | >50 | >50 |
| 3 | Assay | 90-110% | 101.20 | 101.08 |
| 4 | Dissolution | NLT 75(Q)% | 100.68 | 100.22 |
| | Impurities: | | | |
| 5 | Impurity A | NMT 0.5% | 0.08 | 0.10 |
| | Impurity B | NMT 0.5% | ND | ND |
| | Impurity C | NMT 0.8% | ND | ND |
| | Impurity D | NMT 0.5% | ND | ND |
| | Unknown max Impurity | NMT 0.5% | 0.06 | 0.08 |
| | Total Impurities | NMT 1.0% | 0.14 | 0.18 |

ND: Not detected

| S. No. | Ingredients | % w/w |
|---|---|---|
| 1 | Ketorolac tromethamine | 9.61-19.23 |
| 2 | Polyoxyl 40 Hydrogenated Castor Oil | 3.85-7.70 |
| 3 | Sucralose | 2.88-5.77 |
| 4 | Maltodextrin | 4.71-9.42 |
| 5 | Pregelatinized hydroxypropyl pea starch | 2.88-5.77 |
| 6 | Hydroxypropyl Methylcellulose | 15.38-30.77 |
| 7 | Glycerol | 5.76-11.53 |
| 8 | Tartrazine | 0.09-0.19 |
| 9 | Peppermint Flavour | 1.92-3.85 |
| 10 | Orange Flavour | 2.88-5.77 |
| 11 | Purified Water | q.s. |

| | |
|---|---|
| Total | 100.00% |

* water get evaporated during the coating prosses and traces of water content will remain product.

### Manufacturing Procedure

1. Weighed quantity, of purified water was taken in a ss container and kept under stirring at 350 RPM.
2. Weighed quantity of ketorolac tromethamine was added to above step-1 under stirring at 350 RPM for 5 mini.
3. Weighed quantity of Polyoxyl 40 Hydrogenated Castor Oil was added to above step-2 under stirring at 350 RPM for 10 mini.
4. Weighed quantity of sucralose was added to above step-3 under stirring at 350 RPM for 3 min.
5. Weighed quantity of maltodextrin was added to above step-4 under stirring at 350 RPM for 3 min.
6. Weighed quantity of Pregelatinized hydroxypropyl pea starch was added to above step-5 under stirring at 350 RPM for 3 min.
7. Weighed quantity of 11% HPMC solution was added to the above step-6 under stirring at 500 rpm for 10 mins.
8. Weighed quantity of Tartrazine was added to above step-7 under stirring at 500 RPM for 3 min.
9. Weighed quantity of glycerol was added to above step-8 under stirring at 500 RPM for 2 min.
10. Weighed quantity of peppermint Flavour was added to above step-9 under stirring at 500 RPM for 3 min.
11. Weighed quantity of Orange Flavour was added to above step-10 under stirring at 500 RPM for 3 min.
12. Finally, above step-11 Wet slurry was stirred at 500 RPM for 20 min to get uniform dispersion.
13. Above step wet slurry after completion of mixing subjected to vacuum degassing for 15 mins to obtain bubble free slurry.
14. The Wet slurry was casted at process parameters of Dr. Knife thickness of 500 to 600 microns; Temperature 9013° C.; In-direct drying for 15 min.
15. The dried casted film was Slitted into 25 mm×40 mm for 20 mg dose.
16. The dried casted film was slitted into 25 mm×20 mm for 10 mg dose.

The films were evaluated for disintegration time, folding endurance, Assay Dissolution, and impurities. The films were subjected to stability study under 40° C. 2° C. and 75%±5% RH condition for one month and evaluated above test parameters and results are tabulated.

### Results:

| | | | Results | |
|---|---|---|---|---|
| | | | | After |
| S. No. | Test Parameters | Limits | Initial | 1 month |
| 1 | Disintegrating time | NMT 60 sec | 30 sec | 38 sec |
| 2 | Folding Endurance | NLT 10 | >50 | >50 |
| 3 | Assay | 90-110% | 102.00 | 99.02 |
| S. No. | Ingredients | | | % w/w |
| 1 | Ketorolac tromethamine | | | 6.1-12.20 |
| 2 | Polacrilin potassium | | | 9.15-18.30 |
| 3 | Sucralose | | | 1.52-3.05 |
| 4 | Neotame | | | 0.30-0.61 |
| 5 | Maltodextrin | | | 2.27-4.55 |
| 6 | Polyvinylpyrrolidone | | | 1.05-2.10 |
| 7 | Monoammonium Glycyrrhizinate | | | 0.61-1.22 |
| 8 | Polacrilin potassium | | | 3.05-6.10 |
| 9 | Hydroxypropyl Methylcellulose | | | 12.19-24.39 |
| 10 | Triethyl Citrate | | | 3.65-7.31 |
| 11 | Glycerol | | | 6.70-13.41 |
| 12 | Brilliant Blue colour | | | 0.02-0.05 |
| 13 | Capsudex Cool 3MP flavour | | | 0.30-0.61 |
| 14 | Peppermint flavour | | | 3.05-6.10 |
| 15 | Purified Water | | | q.s. |
| Total | | | | 100.00% |
| 4 | Dissolution | NLT 75% | 99.02 | 98.42 |
| | Impurities: | | | |
| 5 | Impurity A | 0.5% | 0.03 | 0.07 |
| | Impurity B | 0.5% | ND | ND |
| | Impurity C | 0.8% | 0.06 | 0.07 |
| | Impurity D | 0.5% | ND | ND |
| | Unknown max | 0.5% | 0.08 | 0.12 |
| | Impurity | | | |
| | Total Impurities | 1.0% | 0.17 | 0.23 |

ND: Not detected

### Example-5

* water get evaporated during the coating prosses and traces of water content will remain product.

### Manufacturing Procedure

1. Weighed quantity of purified water was taken in a ss container and kept under stirring at 350 RPM.
2. Weighed quantity of ketorolac Tromethamine was added to above step-1 under stirring at 350 RPM for 5 min.
3. Weighed quantity of Polacrilin potassium was added to above step-2 under stirring at 400 RPM for 15 min.
4. Weighed quantity of sucralose was added to above step-3 under stirring at 400 RPM for 3 min.
5. Weighed quantity of Neotame was added to above step-4 under stirring at 400 RPM for 3 min.
6. Weighed quantity of maltodextrin was added to above step-5 under stirring at 400 RPM for 5 min.
7. Weighed quantity of Polyvinylpyrrolidone was added to above step-6 under stirring at 400 RPM for 10 min.
8. Weighed quantity of Monoammonium Glycyrrhizinate was added to above step-7 under stirring at 400 RPM for 10 min
9. Weighed quantity of Polacrilin potassium was added to above step-$ under stirring at 400 RPM for 10 min
10. Weighed quantity of 13% HPMC solution was added to the above step-9 under stirring at 500 rpm for 15 mins.
11. Weighed quantity of Brilliant Blue colour was added to above step-10 under stirring at 500 RPM for 3 min.
12. Weighed quantity of Triethyl Citrate was added to above step-11 under stirring at 500 RPM for 5 min.
13. Weighed quantity of glycerol was added to above step-12 under stirring at 500 RPM for 5 min.
14. Weighed quantity of peppermint Flavour was added to above step-13 under stirring at 500 RPM for 3 min.
15. Weighed quantity of Capsudex Cool 3MP was added to above step-14 under stirring at 500 RPM for 3 min.
16. Finally, above step-14 Wet slurry was stirred at 500 RPM for 20 min to get uniform dispersion.
17. Above step wet slurry after completion of mixing subjected to vacuum degassing for 15 mins to obtain bubble free slurry.
18. The Wet slurry was casted at process parameters of Dr. Knife thickness of 600 to 800 microns; Temperature 95±3° C.; In-direct drying for 25 min.
19. The dried casted film was Slitted into 25 mm×40 mm for 20 mg dose.
20. The dried casted film was slitted into 25 mm×20 mm for 10 mg dose.

The films were evaluated for disintegration time, folding endurance, Assay Dissolution, and impurities. The films were subjected to stability study under 40° C.±2° C. and 75%±5% RH condition for one month and evaluated above test parameters and results are tabulated.

### Results:

| | | | Results | |
|---|---|---|---|---|
| S. | | | | After |
| No. | Test Parameters | Limits | Initial | 1 month |
| 1 | Disintegrating time | NMT 60 sec | 20 sec | 24 sec |
| 2 | Folding Endurance | NLT 10 | 40 | 30 |
| 3 | Assay | 90-110% | 100.04 | 99.11 |
| 4 | Dissolution | NLT 75% | 98.02 | 97.00 |
| | Impurities: | | | |
| 5 | Impurity A | 0.5% | 0.08 | 0.16 |
| | Impurity B | 0.5% | ND | ND |
| | Impurity C | 0.8% | 0.08 | 0.08 |
| | Impurity D | 0.5% | ND | ND |
| | Unknown max Impurity | 0.5% | 0.08 | 0.08 |
| | Total Impurities | 1.0% | 0.24 | 0.32 |

ND: Not detected

### CONCLUSION

Based on the experimental data example-2 and example-3 found satisfactory in terms of physical & chemical properties

We claim:

1. An orally disintegrating film composition of ketorolac, wherein the composition comprising of:
a) therapeutically effective concentration of ketorolac of about 10%-20% by total weight of the composition as an active ingredient; Sucralose—0.33%-4% by total weight of the composition; Silicified microcrystalline cellulose 1%-20% by total weight of the composition; Hydroxypropyl Beta-cyclodextrin 0.1%-10% by total weight of the composition; Ammonium Glycyrrhizinate 0.33% 4% by total weight of the composition; Xylitol 0.33%-4% by total weight of the composition; Hydroxyl Propyl Methyl Cellulose 10%-25% by total weight of the composition; Glycerol 0.1%-10% by total weight of the composition; Flavor 2.5%-10% by total weight of the composition; Cooling Agent Flavor 2.5%-10% by total weight of the composition; Sodium Carbonate 0.05%-5% by total weight of the composition; Citric Acid 0.05%-5% by total weight of the composition; Color 0.001%-0.1% by total weight of the composition; Purified Water Q.S to 100% by total weight of the composition and the total strip weight is 100.00%
b) pharmaceutically acceptable excipients that include solubilizers, polymer, plasticizer, disintegrating agent, sweetening agent, cooling agent, flavouring agent, pH modulating agents, colouring agent and purified water.

2. The composition according to claim 1, wherein the solubilizer whose concentration ranges from 0.1%-10% by total weight is selected from the group consisting of benzyl alcohol, hydroxypropyl betacyclodextrin, macrogol 15 hydroxystearate, polyoxyethylene alkyl ethers, polyoxyethylene castor oil derivatives (kolliphor RH 40), povidone, sulfobutylether betacyclodextrin, tricaprylin, triolein, polyoxyethylene sorbitan fatty acid esters, polyethylene-propylene glycol copolymers and any combinations thereof.

3. The process according to claim 1, wherein polymer whose concentration ranges from 10%-25% by total weight is selected from the group consisting of hydroxyl propyl methyl cellulose, ethyl cellulose, hydroxypropyl cellulose, hydroxyl ethyl cellulose, polyvinyl alcohol, cellulose acetate phthalate, hydroxyl propyl methyl cellulose phthalate, polysaccharide derivatives and any combinations thereof.

4. The composition according to claim 1, wherein plasticizer whose concentration ranges from 0.1%-10% by total weight is selected from the group consisting of polyethylene glycol 400, polyethylene glycol 4000, polyethylene glycol 6000, polyethylene glycol 8000, diethyl phthalate, propylene glycol, castor oil and its derivatives, triacetin, triethyl citrate, tributyl citrate, acetyl tributyl citrate, acetyl triethyl citrate, trioctyl citrate, acetyl trioctyl citrate, trihexyl citrate, dibutyl sebacate, polyvinyl pyrrolidone K-30, glycerol, sorbitol, sorbitan derivatives and any combinations thereof.

5. The composition according to claim 1, wherein disintegrating agent whose concentration ranges from 1%-20% by total weight is selected from the group consisting of crospovidone, microcrystalline cellulose, crosscarmellose sodium, Polacrilin potassium, sodium starch glycolate, pregelatinzed starch, silicified microcrystalline cellulose and modified corn starch and any combinations thereof.

6. The composition according to claim 1, wherein sweeting agent whose concentration ranges from 0.33%-4% by total weight is selected from the group consisting of artificial sweeteners like aspartame, sugars derivatives, sucralose, dextrose, fructose, isomalt, lactilol, maltitol, ammonium glycyrrhizinate, maltose, mannitol, sorbitol, Aspartame, sodium saccharine, Acesulfame Potassium, Stevioside, starch hydrolysate, polydextrose and xylitol and any combinations thereof.

7. The composition according to claim 1, wherein cooling agent whose concentration ranges from 2.5%-10% by total weight is selected from the group consisting of peppermint flavour, cooling flavour (menthol), flavour oils, flavouring aromatic oil, peppermint oil, clove oil, bay oil, anise oil, eucalyptus oil thyme oil, oil of bitter almonds and any combinations thereof.

8. The composition according to claim 1, flavouring agent whose concentration ranges from 2.5%-10% by total weight is selected from the group consisting of vanilla, chocolate flavour, citrus oils, fruit essences, and any combinations thereof.

9. The composition according to claim 1, wherein pH modulating agent whose concentration ranges from 0.05%-5% by total weight is selected from the group consisting of sodium carbonate, ammonium carbonate, sodium bicarbonate, potassium bicarbonate, calcium carbonate, magnesium carbonate, disodium glycine carbonate, sodium glycine carbonate, lysine carbonate, arginine carbonate, citric acid, tartaric acid, malic acid, fumaric acid, metatartaric acid, adipic acid, sodium acid citrate, ascorbic acid and amino acids such as aspartic acid, glutamic acid, glycine, leucine, tyrosine and tryptophan and any combinations thereof.

10. The composition according to claim 1, wherein colouring agent whose concentration ranges from 0.001%-0.1% by total weight is selected from the group consisting of sunset yellow, amaranth, red iron oxide, natural juice concentrates, pigments and opacifying agents such as titanium oxide, silicon dioxide and zinc oxide, solid choco color and any combinations thereof.
